(19) United States
(12) Patent Application Publication
(10) Pub. No.: US 2026/0199129 A1
(43) Pub. Date: Jul. 16, 2026

(54) Z-KAP SCLERAL PROTECTOR FOR TREATMENT OF CONJUNCTIVA WITH ANTIMETABOLITES AND OTHER THERAPEUTICS DURING OPHTHALMIC SURGICAL PROCEDURES

(71) Applicant: UNIVERSITY OF MIAMI, Miami, FL (US)

(72) Inventors: Carol Karp, Miami Beach, FL (US); Jeffrey Peterson, Chicago, IL (US); Diego E. Alba, Miami, FL (US); Mike Zein, Chicago, IL (US)

(21) Appl. No.: 19/139,259

(22) PCT Filed: Dec. 19, 2023
(86) PCT No.: PCT/US2023/084749

§ 371 Date: Jun. 13, 2025

Related U.S. Application Data
(60) Provisional application No. 63/435,503, filed on Dec. 27, 2022.

(51) Int. Cl. A61F 9/00; A61F 9/007

(52) U.S. Cl. CPC A61F 9/0017; A61F 9/007

(57) ABSTRACT

A scleral protector device which allows for controlled and safe application of antimetabolite during conjunctival lesion excision surgery.

### FIELD

This disclosure relates to devices and methods for the safe application of MMC, 5-fluorouracil, or other antimetabolite onto the conjunctiva with simultaneous protection of the exposed sclera during conjunctival tumor or pterygium excision surgery.

### BACKGROUND

Conjunctival malignancies and pterygium affect a large number of patients worldwide. Pterygia are fibrous growths over the surface of the eye which can result in distorted and decreased vision, irritation, and even restriction of motility, with a worldwide prevalence of 10.2%. These lesions are due to UV exposure and are especially common in the sunbelt zones and those with outdoor work or hobbies. Studies have shown that pterygia removed with the bare sclera surgical technique has 24-89% recurrence. To prevent this recurrence, the conjunctiva is treated with cryotherapy and/or antimetabolite therapy during the surgical procedure. Conjunctival malignancies are less common than pterygia, with the most common ocular surface tumor, ocular surface squamous neoplasia (OSSN), affecting 0.01 to 3.4 per 100 000 persons/year. While more rare, untreated OSSN can lead to blindness or even death. Similar to pterygium excision, recurrence rates of OSSN after surgery can be high, ranging from 5% to 69%. Thus, application of antimetabolite and/or cryotherapy during OSSN excision is also practiced to reduce recurrence.

Mitomycin C (MMC) is an antimetabolite derived from Streptotomyces caespitosus which has anti-neoplastic properties used to treat a variety of cancers and benign growths on the eye. It is a commonly used adjuvant in conjunctival tumor and pterygium excision surgery to prevent recurrence (24-89% recurrence without MMC) and to eliminate any underlying subclinical disease. Studies have shown MMC to reduce recurrence of pterygium by 2.75× to 16×, or an absolute reduction of recurrence by 24-45% percent. While it reduces risk of ocular surface tumor recurrence, MMC use brings with it a number of complications, which include the risk of scleral melt or perforation. Consequences of scleral melting from anti-metabolite exposure can include infection, scleritis, and even loss of the eye. Therefore, when treating the conjunctival tissue with MMC during conjunctival tumor or pterygium excision, the sclera requires adequate protection. To date, there is no known device specifically available for this purpose.

Currently, there is no standard method for the safe application of MMC, 5-fluorouracil, or other antimetabolite onto the conjunctiva with simultaneous protection of the exposed sclera during conjunctival tumor or pterygium excision surgery. One method involves soaking a sponge with MMC and retracting the conjunctival flap away from the sclera and pressing the MMC soaked sponge against the inner conjunctival surface. This current methodology has several problems, including that it is not well standardized, as well that it allows for easy leakage of MMC from the sponge down onto the scleral tissue, as evidenced in FIG. 3. At our institution, we have adopted an alternative approach which involves using a flat piece of sterile plastic repurposed as a scleral shield. However, this method too lacks standardization and may be prone to leakage. We propose a more controlled method for application of MMC, as well as other antimetabolites or other therapies which require application to the conjunctiva while avoiding scleral contact. We propose a scleral protector device which allows for this controlled and safe application of antimetabolite during conjunctival lesion excision surgery.

### SUMMARY

We propose a device and standardized methodology for applying MMC, or other antimetabolite/therapeutic agent to the conjunctiva during ocular surface tumor and/or pterygium removal, to provide precise treatment while reliably avoiding exposure of the sclera to the therapeutic agent. This novel surgical device we call the Z-KAP (Zein-Karp-Alba-Peterson) scleral protector (see FIGS. 1 and 2). During ocular surface tumor removal, the conjunctiva is separated and retracted away from the scleral surface after the tumor is removed. The Z-KAP device is placed underneath the retracted conjunctival flap, over the exposed sclera. The Z-KAP scleral protector has an easy to use, ergonomic, angled handle with which the surgeon can comfortably maneuver the device into place. The Z-KAP scleral protector contains a scleral protector element, which consists of a smooth bottomed, snugly fitting basin which is placed directly onto the sclera. This scleral protector element is curved to match the curvature of the sclera (11 mm radius of curvature in this iteration, but this can be changed to match different curvatures depending on patient eye). On the top side of the protective element is a lipped basin in which to place the antimetabolite-soaked sponge.

Once the scleral protector and sponge are in place, the retracted conjunctiva is folded back over the sponge and basin for a desired length of time, during which antimetabolite from the sponge treats the overlying conjunctiva. It is during this time that the basin lip makes contact with the conjunctiva to create a seal, which prevents the antimetabolite from leaking out onto the sclera underneath.

For the prototype described in this document, the Z-KAP scleral protector is 3D printed from polylactic acid. However, it can be constructed from any number of materials, such as stainless steel or a medically safe plastic.

We tested the efficacy of the scleral protector device against other methods as described in FIG. 6. The unique features of this device include the curvature molding to the scleral surface, and the lipped basin which limits egress of the chemotherapy fluids onto the sclera.

### BRIEF DESCRIPTION OF THE DRAWINGS

FIG. 1 is a perspective view of a scleral protector of the present disclosure;

FIG. 2 is a top perspective view of a pair of scleral protectors of the present disclosure;

FIG. 3 is a side view of the scleral protector of FIG. 1;

FIG. 4 is an image illustrating the scleral protector of FIG. 1 in use in a surgical procedure;

FIG. 5 is an image illustrating the scleral protector of FIG. 1 in use in a surgical procedure, following placement of a sponge on top of a sponge basin of the scleral protector, with a conjunctival flap retracted over the sponge;

FIG. 6 is a series of images comparing different methods of performing surgical procedures utilizing a scleral protector of the present disclosure.

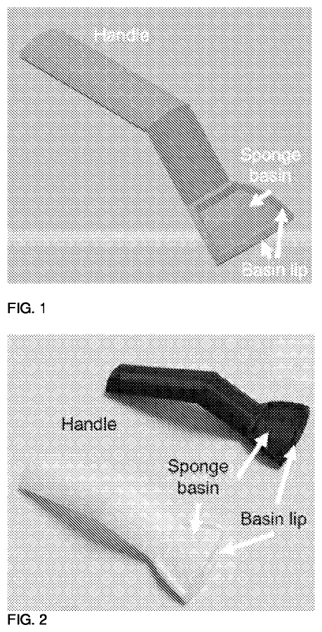

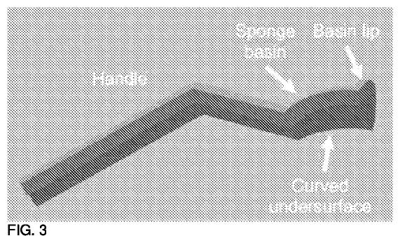

### DETAILED DESCRIPTION OF THE PREFERRED EMBODIMENTS

Conjunctival malignancies and pterygium affect a large number of patients worldwide. Pterygia are fibrous growths over the surface of the eye which can result in distorted and decreased vision, irritation, and even restriction of motility, with a worldwide prevalence of 10.2%. These lesions are due to UV exposure and are especially common in the sunbelt zones and those with outdoor work or hobbies. Studies have shown that pterygia removed with the bare sclera surgical technique has 24-89% recurrence. To prevent this recurrence, the conjunctiva is treated with cryotherapy and/or antimetabolite therapy during the surgical procedure. Conjunctival malignancies are less common than pterygia, with the most common ocular surface tumor, ocular surface squamous neoplasia (OSSN), affecting 0.01 to 3.4 per 100 000 persons/year. While more rare, untreated OSSN can lead to blindness or even death. Similar to pterygium excision, recurrence rates of OSSN after surgery can be high, ranging from 5% to 69%. Thus, application of antimetabolite and/or cryotherapy during OSSN excision is also practiced to reduce recurrence.

Mitomycin C (MMC) is an antimetabolite derived from Streptotomyces caespitosus which has anti-neoplastic properties used to treat a variety of cancers and benign growths on the eye. It is a commonly used adjuvant in conjunctival tumor and pterygium excision surgery to prevent recurrence (24-89% recurrence without MMC) and to eliminate any underlying subclinical disease. Studies have shown MMC to reduce recurrence of pterygium by 2.75× to 16×, or an absolute reduction of recurrence by 24-45% percent. While it reduces risk of ocular surface tumor recurrence, MMC use brings with it a number of complications, which include the risk of scleral melt or perforation.

Consequences of scleral melting from anti-metabolite exposure can include infection, scleritis, and even loss of the eye. Therefore, when treating the conjunctival tissue with MMC during conjunctival tumor or pterygium excision, the sclera requires adequate protection. To date, there is no known device specifically available for this purpose.

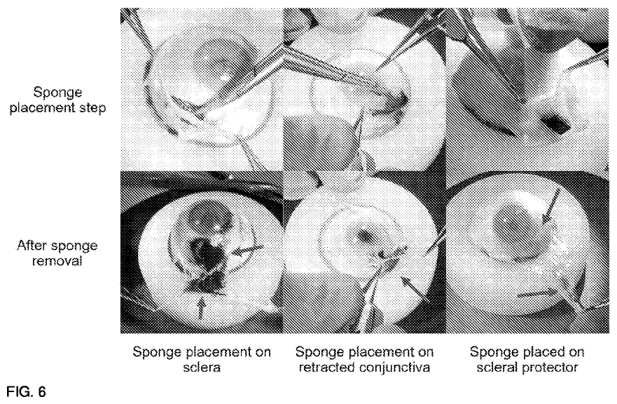

Currently, there is no standard method for the safe application of MMC, 5-fluorouracil, or other antimetabolite onto the conjunctiva with simultaneous protection of the exposed sclera during conjunctival tumor or pterygium excision surgery. One method involves soaking a sponge with MMC and retracting the conjunctival flap away from the sclera and pressing the MMC soaked sponge against the inner conjunctival surface. This current methodology has several problems, including that it is not well standardized, as well that it allows for easy leakage of MMC from the sponge down onto the scleral tissue, as evidenced in FIG. 6. At our institution, we have adopted an alternative approach which involves using a flat piece of sterile plastic repurposed as a scleral shield. However, this method too lacks standardization and may be prone to leakage. We propose a more controlled method for application of MMC, as well as other antimetabolites or other therapies which require application to the conjunctiva while avoiding scleral contact.

We propose a scleral protector device which allows for this controlled and safe application of antimetabolite during conjunctival lesion excision surgery.

The unique features of this device include the curvature molding to the scleral surface, and the lipped basin which limits egress of the chemotherapy fluids onto the sclera. No other such dedicated device currently exists for antimetabolite application during pterygium or ocular surface tumor excision.

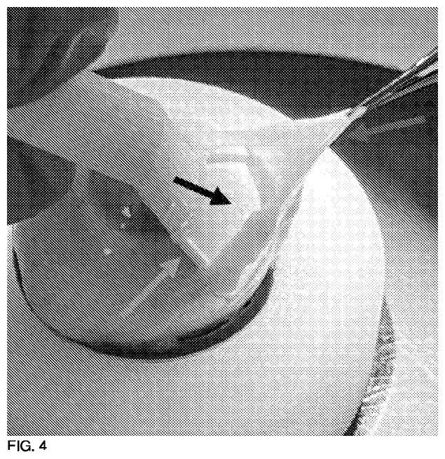

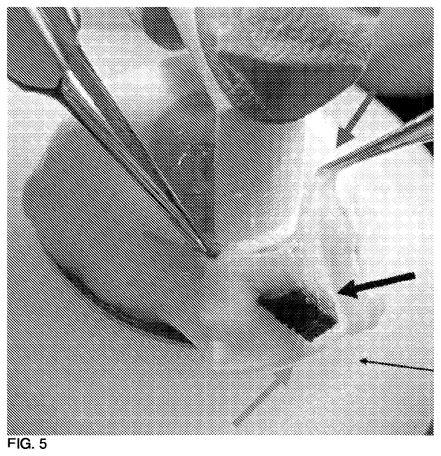

As illustrated in FIG. 4, once the ocular surface tumor or pterygium has been removed and the conjunctival flap has been retracted, the sclera protector (blue and black arrows) is placed over the exposed sclera and under the retracted conjunctival flap (red arrow). Once cut to an appropriate size, the sponge is soaked with the therapeutic agent of choice (for example, an antimetabolite such as mitomycin C) and placed on top of the lipped basin of the scleral protector (black arrow). In this example however, the sponge was soaked with black tissue dye for better visualization. As illustrated in FIG. 5, once the sponge has been placed on top of the sponge basin of the scleral protector (black arrow), the conjunctival flap is retracted over the sponge, which creates gentle pressure on the sponge to release the therapeutic agent. Contact between the sponge basin lip and the conjunctiva (blue arrow) creates a seal which prevents leakage of the therapeutic agent.

### EXAMPLES

We tested the efficacy of the scleral protector device against other methods, using three treatment groups: Group 1 (n=3): sponge placed directly on sclera, with conjunctiva pulled over the sponge, Group 2 (n=3): sponge held against deep conjunctival surface, away from exposed sclera, Group 3 (n=4): sponge placed on scleral protector and conjunctiva pulled back over both sponge and protector.

An ophthalmic surgical sponge was cut to a size that fits well within the lipped sponge basin (in this iteration 4×6×2 mm after soaking). Note that the depth dimension of the sponge must be shallow enough to allow for creation of a seal between the basin lip and conjunctiva. The surgical sponge was soaked with 1:1000 black tissue marking dye in water (Mfr. No., 07271, Cancer Diagnostics, Inc., Durham, NC).

Group 1 & 2 had significant staining on the scleral and conjunctival surface, as well as 2/4 eyes in the scleral protector group. Two of four eyes in the scleral protector group had no scleral staining (see above), with only staining on the conjunctiva.

The two leaks seen using the scleral protector were attributed to 1) placing the sponge on the conjunctiva first instead of directly onto the protector 2) and cutting the sponge such that it was too thick (>6 mm, when ideal thickness is <4 mm after soaking), preventing proper sealing between the conjunctiva and the basin lip. Proper technique ensured that 2/4 eyes tested with the scleral protector had no ink leakage.

1. A scleral protector device comprising a handle, a sponge basin at a distal end of the handle, and a basin lip surrounding a perimeter of the sponge basin.

2. The scleral protector device of claim 1, the sponge basin being convex, defining a curved and concave undersurface.

3. The scleral protector device of claim 1 the basin lip having a rounded upper end.

4. The scleral protector device of claim 1, the handle being curved.

5. The scleral protector device of claim 1, the basin lip including a scleral protector element curved to match the curvature of the sclera of an eye of a patient.

6. The scleral protector device of claim 6, the scleral protector element having an 11 mm radius of curvature.
